(19) United States
(12) Patent Application Publication
(10) Pub. No.: US 2025/0025174 A1
(43) Pub. Date: Jan. 23, 2025

(54) ORTHOPEDIC IMPLANTS AND INSTRUMENTS

(71) Applicant: Paragon 28, Inc., Englewood, CO (US)

(72) Inventors: Peter Andrew MLADINICH, Parker, CO (US); Kenneth Allan ROGGOW, Denver, CO (US); Kaitlin KARAS, Denver, CO (US); Benjamin MAJORS, Denver, CO (US)

(73) Assignee: Paragon 28, Inc., Englewood, CO (US)

(21) Appl. No.: 18/907,097

(22) Filed: Oct. 4, 2024

Related U.S. Application Data
(63) Continuation of application No. PCT/US2023/065390, filed on Apr. 5, 2023.
(60) Provisional application No. 63/362,569, filed on Apr. 6, 2022.

(51) Int. Cl. A61B 17/10; A61B 17/00; A61B 17/064

(52) U.S. Cl. CPC A61B 17/10; A61B 17/0642; A61B 2017/00473; A61B 2017/0645

(57) ABSTRACT

An orthopedic implant system includes an insertion instrument having an actuator and a retention mechanism. The implant system also includes an orthopedic staple implant configured to releasably couple with the retention mechanism of the insertion instrument and, upon actuation of the actuator, decouple from the insertion instrument. Also disclosed is a surgical instrument having an upper portion and a lower portion that is integral with the upper portion. The lower portion includes an actuator extending laterally and a pair of retainers extending from the lower portion. Each retainer has a substantially curved geometry with the curvature pointing toward a midline of the instrument. An orthopedic staple implant is also disclosed that has a bridge and a pair of legs extending from a bottom surface of the bridge. Each of the legs includes a texture or gripping configuration disposed on an inner surface of the leg.

### CROSS REFERENCE TO RELATED APPLICATION

This application is a continuation of International Patent Application No. PCT/US2023/065390 filed Apr. 5, 2023 and entitled “Orthopedic Implants and Instruments,” which claims priority benefit under 35 U.S.C. § 1119(e) to U.S. Provisional Patent Application No. 63/362,569, filed on Apr. 6, 2022, and entitled “Orthopedic Insertion Instruments,” which are incorporated herein by reference in their entireties.

### TECHNICAL FIELD

The present disclosure relates to implants, devices, and methods associated with performing orthopedic procedures. The present disclosure relates to podiatric and orthopedic implants and surgery related to arthroplasty, arthrodesis, and/or arthroeresis of joints in the foot/ankle and/or procedures incorporating surrounding bones/soft tissue.

### BACKGROUND OF THE INVENTION

Many currently available implants, devices, and methods for addressing bone, soft tissue, and joint trauma (acute and chronic, e.g., defect, gradual deterioration, etc.) do not completely address the needs of patients. Additionally, many currently available implants, devices, and methods for addressing joint trauma fail to account for properties of joint anatomy and associated mechanical and kinematic movement patterns/capabilities.

### SUMMARY OF THE INVENTION

A first aspect of the present disclosure is directed toward an orthopedic implant system. The system includes an insertion instrument having an actuator and a retention mechanism. The implant system also includes an implant configured to releasably couple with the retention mechanism of the insertion instrument and, upon actuation of the actuator, decouple from the insertion instrument.

According to the first aspect of the present disclosure, the insertion instrument includes an upper portion and a lower portion, wherein the upper and lower portions are integral with one another.

According to the first aspect of the present disclosure, the actuator is integral with the lower portion of the insertion instrument.

According to the first aspect of the present disclosure, the actuator extends laterally from the lower portion of the insertion instrument.

According to the first aspect of the present disclosure, the retention mechanism extends from the lower portion of the insertion instrument and includes a pair of retainers.

According to the first aspect of the present disclosure, each of the retainers is positioned at an opposite side of the bottom portion of the insertion instrument.

According to the first aspect of the present disclosure, the insertion instrument has a volume defined vertically by the lower portion of the insertion instrument and laterally by the retainers.

According to the first aspect of the present disclosure, each of the retainers has a substantially curved geometry, wherein said curved geometry points toward the volume.

According to the first aspect of the present disclosure, the volume is configured to receive and retain at least a portion of the implant therein.

According to the first aspect of the present disclosure, manipulation of the actuator is configured to manipulate at least one of the retainers so as to couple and decouple the implant from the insertion instrument.

According to the first aspect of the present disclosure, the implant includes an orthopedic staple.

A second aspect of the present disclosure includes a surgical instrument that includes an upper portion and a lower portion. The lower portion is integral with and opposite from the upper portion. The lower portion includes an actuator extending laterally from the lower portion, and a pair of retainers extending from a bottom portion of the lower portion toward the upper portion, wherein each of the retainers has a substantially curved geometry with said curvature pointing toward a midline of the instrument

According to the second aspect of the present disclosure, the instrument includes a first volume defined at least partially by a lateral surface of the instrument and a lateral surface of the actuator.

According to the second aspect of the present disclosure, the actuator is manipulatable such that at least a portion of the actuator occupies at least a portion of the first volume.

According to the second aspect of the present disclosure, the actuator is pivotable relative to the lower portion of the instrument.

According to the second aspect of the present disclosure, manipulation of the actuator alters the lateral dimension of a second volume defined laterally by the retainers.

A third aspect of the present disclosure includes an orthopedic staple implant. The implant includes a bridge and a pair of legs extending from a bottom surface of the bridge, with each of the legs including a texture on an inner surface thereof.

According to the third aspect of the present disclosure, the implant is movable between a first position comprising the bridge in a substantially curved configuration, and a second position comprising the bridge in a substantially linear configuration.

According to the third aspect of the present disclosure, each of the legs extend from the bottom surface of the bridge at a point between a midline of the bridge and a terminal end point of the bridge.

According to the third aspect of the present disclosure, each of the legs include a tapered thickness, wherein said thickness is greater at a point adjacent the bridge than at a point adjacent the terminal end of the legs.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

FIG. 1 is a front view of an orthopedic implant system including an orthopedic instrument and an orthopedic implant, in accordance with the present disclosure;

FIG. 2 is a rear view of the orthopedic implant system of FIG. 1 including an orthopedic instrument and an orthopedic implant, in accordance with the present disclosure;

FIG. 3 is a front perspective view of the instrument of the system of FIG. 1, in accordance with the present disclosure;

FIG. 4 is a rear perspective view of the instrument of FIG. 3 of the system of FIG. 1, in accordance with the present disclosure;

FIG. 5 is a bottom perspective view of the instrument of FIG. 3 of the system of FIG. 1, in accordance with the present disclosure;

FIG. 6 is a first side view of the instrument of FIG. 3 of the system of FIG. 1, in accordance with the present disclosure;

FIG. 7 is a left side view of the instrument of FIG. 3 of the system of FIG. 1, in accordance with the present disclosure;

FIG. 8 is a top view of the instrument of FIG. 3 of the system of FIG. 1, in accordance with the present disclosure;

FIG. 9 is a bottom view of the instrument of FIG. 3 of the system of FIG. 1, in accordance with the present disclosure;

FIG. 10 is a side perspective view of the instrument of FIG. 3 of the system of FIG. 1, in accordance with the present disclosure;

FIG. 11 is a front view of an orthopedic implant system including an orthopedic instrument and an orthopedic implant, in accordance with the present disclosure;

FIG. 12 is a rear view of the orthopedic implant system of FIG. 11 including an orthopedic instrument and an orthopedic implant, in accordance with the present disclosure;

FIG. 13 is a front view of an orthopedic implant system including an orthopedic instrument and an orthopedic implant, in accordance with the present disclosure;

FIG. 14 is a rear view of the orthopedic implant system of FIG. 13 including an orthopedic instrument and an orthopedic implant, in accordance with the present disclosure

FIG. 15 is a front view of an orthopedic implant system including an orthopedic instrument and an orthopedic implant, in accordance with the present disclosure;

FIG. 16 is a rear view of the orthopedic implant system of FIG. 15 including an orthopedic instrument and an orthopedic implant, in accordance with the present disclosure;

FIG. 17 is a front view of an orthopedic implant system including an orthopedic instrument and an orthopedic implant, in accordance with the present disclosure;

FIG. 18 is a rear view of the orthopedic implant system of FIG. 17 including an orthopedic instrument and an orthopedic implant, in accordance with the present disclosure;

FIG. 19 is a front view of an orthopedic implant system including an orthopedic instrument and an orthopedic implant, in accordance with the present disclosure;

FIG. 20 is a rear view of the orthopedic implant system of FIG. 19 including an orthopedic instrument and an orthopedic implant, in accordance with the present disclosure;

FIG. 21 is a front view of an orthopedic implant system including an orthopedic instrument and an orthopedic implant, in accordance with the present disclosure;

FIG. 22 is a rear view of the orthopedic implant system of FIG. 21 including an orthopedic instrument and an orthopedic implant, in accordance with the present disclosure;

FIG. 23 is a front perspective view of an orthopedic implant, in accordance with the present disclosure;

FIG. 24 is a rear perspective view of the orthopedic implant of FIG. 23, in accordance with the present disclosure

FIG. 25 is a side perspective view of the orthopedic implant of FIG. 23, in accordance with the present disclosure;

FIG. 26 is a side view of the orthopedic implant of FIG. 23, in accordance with the present disclosure;

FIG. 27 is a bottom view of the orthopedic implant of FIG. 23, in accordance with the present disclosure;

FIG. 28 is a bottom perspective view of the orthopedic implant of FIG. 23, in accordance with the present disclosure;

FIG. 29 is an alternate bottom perspective view of the orthopedic implant of FIG. 23, in accordance with the present disclosure; and

FIG. 30 is a top view of the orthopedic implant of FIG. 23, in accordance with the present disclosure.

### DETAILED DESCRIPTION OF THE INVENTION

In this detailed description and the following claims, the words proximal, distal, anterior, or plantar, posterior, or dorsal, medial, lateral, superior, and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers the bottom of the foot.

Similarly, positions or directions may be used herein with reference to anatomical structures or surfaces. For example, as the current implants, devices, instrumentation, and methods are described herein with reference to use with the bones of the foot, the bones of the foot, ankle and lower leg may be used to describe the surfaces, positions, directions or orientations of the implants, devices, instrumentation, and methods. Further, the implants, devices, instrumentation, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation, and methods, and the aspects, components, features and the like thereof, described herein with respect to the right foot may be mirrored so that they likewise function with the left foot. Further, the implants, devices, instrumentation, and methods, and the aspects, components, features and the like thereof, disclosed herein are described with respect to the foot for brevity purposes, but it should be understood that the implants, devices, instrumentation, and methods may be used with other bones of the body having similar structures.

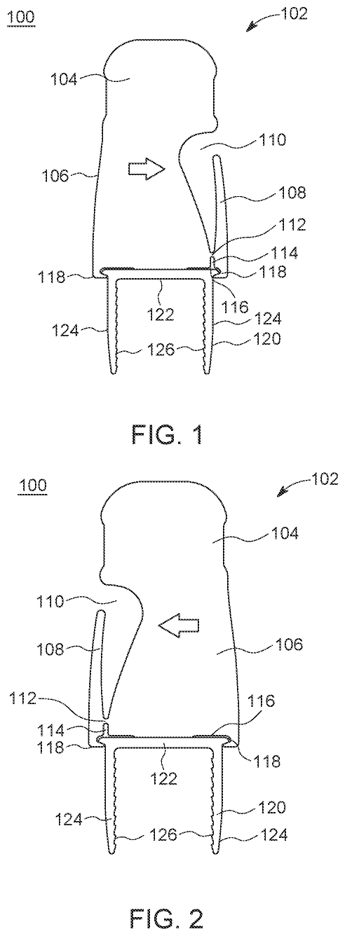

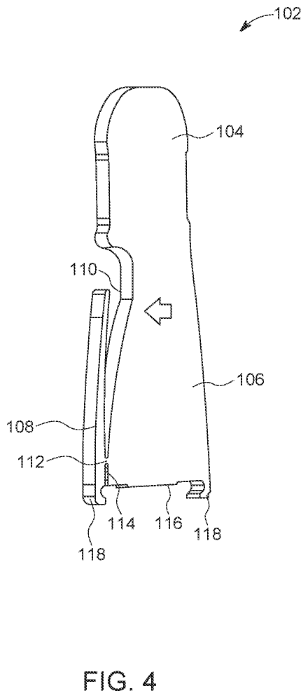

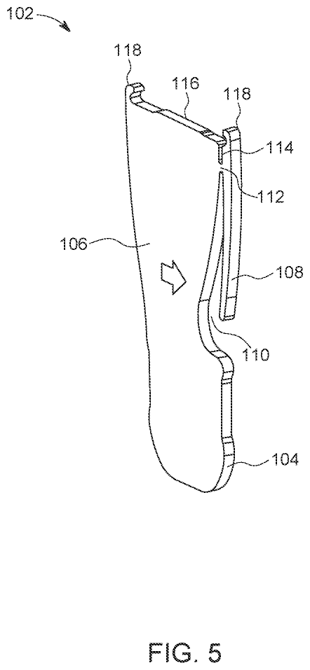

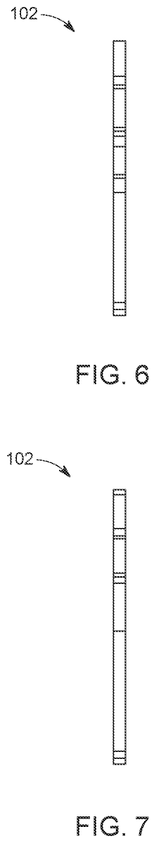

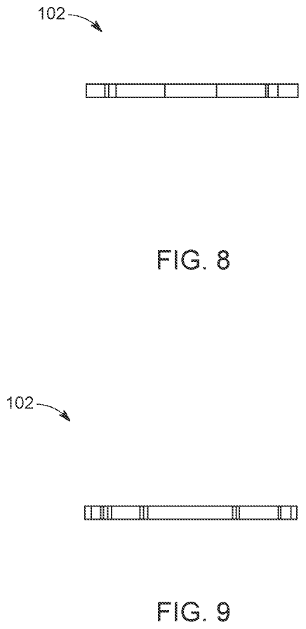

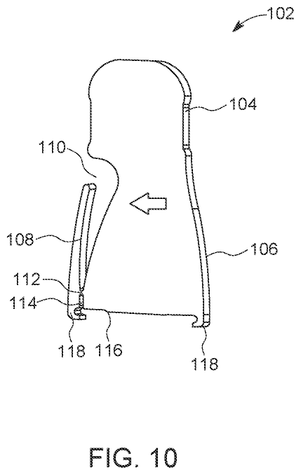

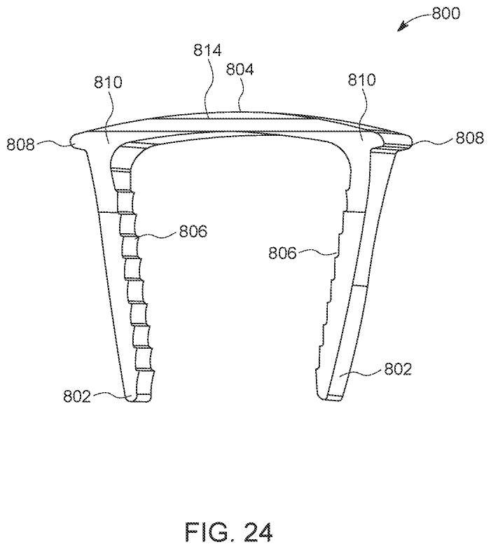

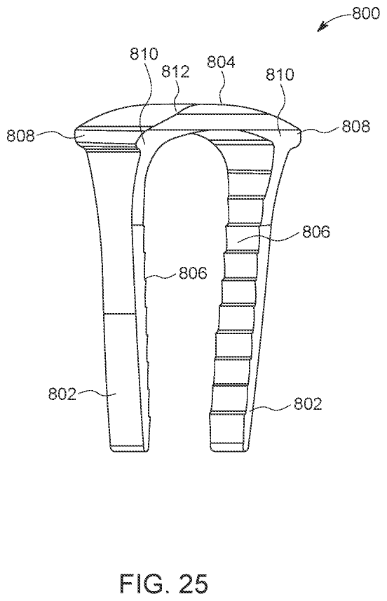

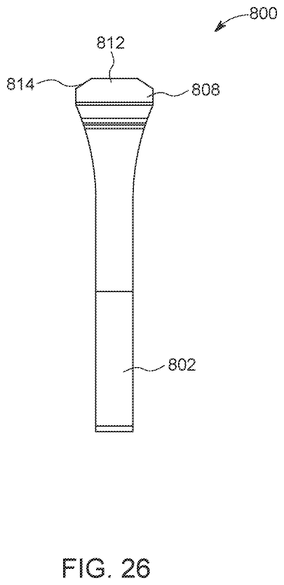

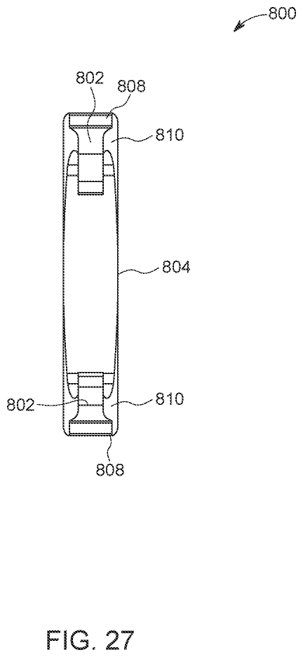

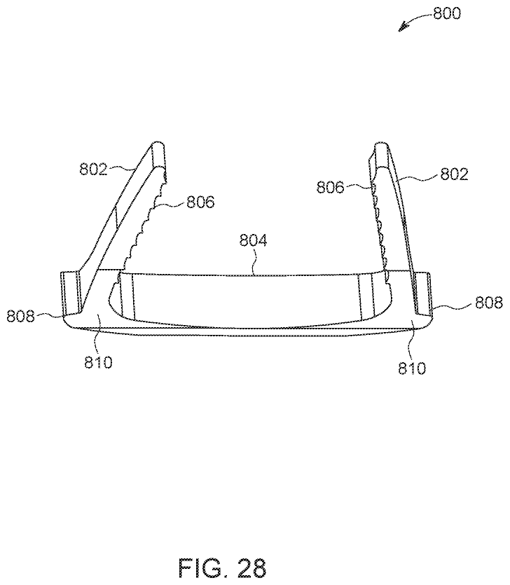

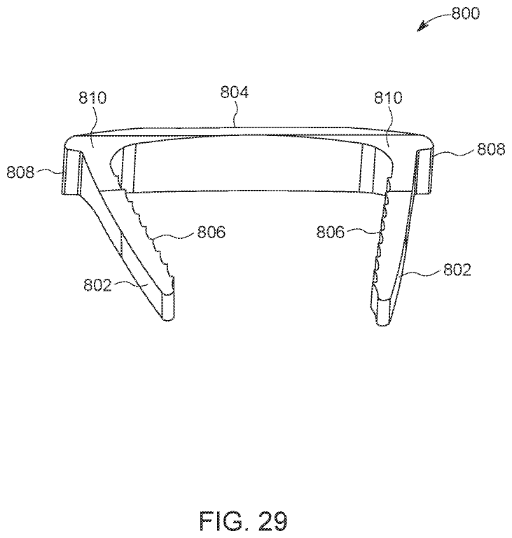

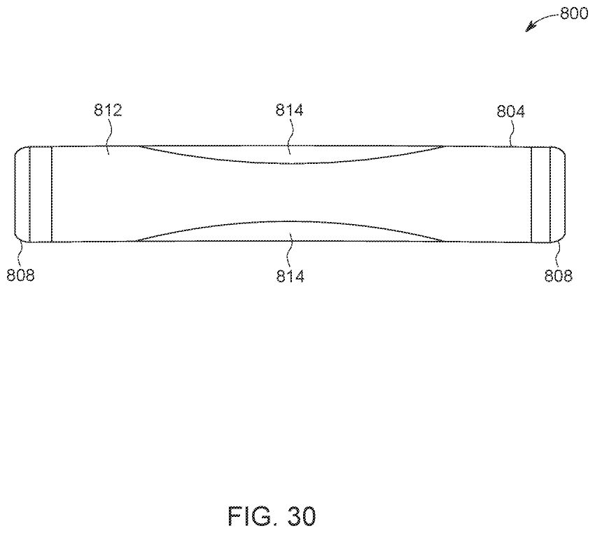

Referring to the drawings, wherein like reference numerals are used to indicate like or analogous components throughout the several views, and with particular reference to FIGS. 1-30, orthopedic implant systems, instruments, and implants are shown. The implant systems, instruments, and implants shown may be cross-compatible. For example, one or more components of a system shown and described herein may be compatible and/or interchangeable with one or more components of an alternative system and/or instruments/implants as shown herein. Further, an implant (e.g., a staple) may be compatible with one or more instruments (e.g., inserters) including but not limited to those shown and described herein. Similarly, an instrument (e.g., an inserter) may be compatible with one or more implants (e.g., staples) including but not limited to those shown and described herein.

Referring now to FIGS. 1-10, an implant system 100 is shown, according to an exemplary embodiment. The implant system 100 may be variously sized, for example with one or more components configured in various sizes and the complementary component configured in a complementary size (e.g., a staple implant in various height/width configurations and an insertion instrument configured to releasably couple with said staple implant).

The system 100 is shown to include an insertion instrument 102 (referred to hereinafter as “inserter 102”), which is releasably couplable with an implant 120 (e.g., a staple) (referred to hereinafter as “staple 120”). The inserter 102 and the staple 120 may be provided to a physician or other healthcare provider/facility in a coupled configuration (e.g., as shown in FIGS. 1-2), or may be provided in a decoupled configuration. If provided in a decoupled configuration, a physician or other healthcare provider may releasably couple (e.g., engage, etc.) the inserter 102 with the staple 120 prior to use or performing any procedure in which the system 100 and components thereof may be implemented. Further, the inserter 102 may be configured to releasably couple and decouple with multiple staples 102 in successive fashion, and as such may be configured to be a multi-use instrument. For example, a physician may releasably couple a first staple 120 with the inserter, decouple and implant said first staple 120, subsequently releasably couple a second staple 120 with the inserter 102, and ultimately decouple and implant said second staple 120.

The inserter 102 is shown to include an upper portion 104 configured substantially opposite a lower portion 106. As shown, the staple 120 is configured to releasably couple with a portion of the lower portion 106 of the staple 120, which is to say that the upper portion 104 is configured opposite the inserter 102 from the coupling site of the staple 120. In some aspects, one or both of the upper and lower portions 104, 106 of the inserter 102 may include one or more ergonomic features, for example geometric contours configured to facilitate gripping and manipulating/actuating the inserter 102 and components thereof. As shown, the upper and lower portions 104, 106 are integral with one another. However, in some aspects, the upper and lower portions 104, 106 may be releasably, hingedly, or otherwise coupled with one another.

The lower portion 106 is shown to include an actuator 108 extending laterally from a side of the lower portion 106. As shown, the actuator 108 is integral with the lower portion 106, but is movable relative to the lower portion 106. The actuator 108 includes an elongated geometry extending from the lower portion 106 and defining a volume (e.g., a space, opening, etc.) 110 positioned between at least a portion of the actuator 108 and the lower portion 106. The actuator 108 is manipulatable through a range of motion that includes at least the position shown in FIGS. 1-2 (an unactuated position), and a position in which at least a portion of the actuator 108 occupies at least a portion of the volume 110 (an actuated position). The actuator 108 may be formed from a resilient material and/or be structured such that a force must be applied to manipulate the actuator 108 from the unactuated position to the actuated position. Further, a force may be required to retain the actuator 108 in the actuated position, where absence of such a force may result in the actuator 108 returning to the unactuated position.

The actuator 108 is configured to be manipulatable about a pivot point 112 (referred to hereinafter as “point 112”) (or, in some aspects, a hinge point), where said point 112 is positioned at or adjacent to the point on the lower portion 106 as which the actuator 108 extends laterally. In some aspects, the point 112 may be configured on a portion of the lower portion that has a lesser width (e.g., lateral dimension) than the actuator 108. Further, the point 112 is positioned substantially below the volume 110 and above a volume 114, where the volume 114 is configured to receive at least a portion of the actuator 108 as said actuator 108 moves through the aforementioned range of motion. The volumes 110, 114 are both configured to fall within the footprint of the inserter 102.

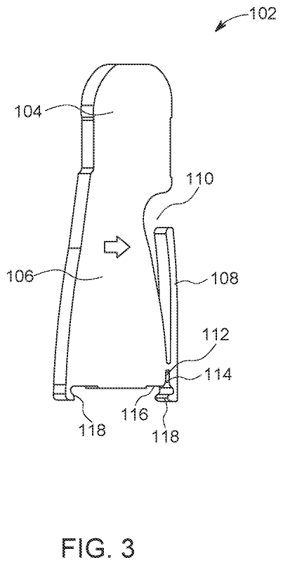

The lower portion 106 is further shown to include a volume 116, which is defined in an upward direction by the lower portion 106 and in lateral directions by a pair of retainers 118. Each of the retainers 118 are shown to extend downward from a bottom portion of the lower portion 106, and are further shown to include substantially curved or “hooked” geometries with said curvature in the direction of a midline of the inserter 102 (e.g., toward the volume 116). The volume 116 as shown is not bound on a bottom side (e.g., opposite the portion defined by the bottom portion of the lower portion 106 when the inserter 102 is in an uncoupled state relative to the staple 120 (e.g., as shown in FIG. 3). As shown, the volume is shown to have an elongated geometry (as defined on three sides), with a width dimension greater than a height dimension.

As shown in FIGS. 1-2, the volume 116 is shown to receive a bridge 122 of the staple 120 when the staple 120 is in a coupled configuration with the inserter 102. In some aspects, the staple 102 may be manipulated from a first position (e.g., with the bridge 122 in a substantially curved geometry) to a second position (e.g., with the bridge 122 in a substantially straight geometry) prior to coupling with the inserter 102. In some aspects, a force may be required to manipulate the staple 102 and the bridge 122 thereof from the first position to the second position. As shown in FIGS. 1-2, said coupling includes positioning the bridge 122 of the staple 120 (in the aforementioned substantially straight geometry) at least partially within the volume 116 of the inserter 102.

The staple 120 is shown to include a pair of legs 124 extending from the bridge 122 at an angle substantially perpendicular to a bottom surface of the bridge 122. In the first aforementioned position, the legs 124 may be positioned at a substantially oblique angle relative to one another. However, in the second position (e.g., the position the staple 120 is retained in when coupled with the inserter 102), the legs 124 may be substantially parallel to one another. The legs 124 are further shown to include a texture 126 disposed on an inner surface thereof. As shown in FIGS. 102, the texture 126 includes a serration or toothed configuration, although in some aspects the texture 126 may also include alternate geometries or configurations.

As shown in FIGS. 1-2, the legs 124 extend downward from the bottom surface of the bridge 122 at points adjacent to the terminal ends of the bridge 122. However, each end of the bridge 122 is configured to extend laterally past the point from which the legs 124 extend. The aforementioned end portions of the bridge 122 (e.g., overhangs, shoulders, etc.) are configured to interface with the retainers 118 of the inserter 102 when in the coupled position shown in FIGS. 1-2. In coupling the staple 120 with the inserter 102, the staple may be manipulated to the second position (substantially straight/linear bridge), with the bridge 122 portion then inserted into the volume 116 such that the end portions of the bridge 122 abut the retainers 118 such that the retainers 118 exert a force on the end portions of the bridge 122 so as to retain the bridge 122 in the substantially straight/linear position.

Upon insertion, the actuator 108 of the inserter 102 may be manipulated such that at least a portion of the actuator 108 occupies at least a portion of the volume 110 and, accordingly, retainer 118 integral with the actuator 108 disengages from the end portion of the bridge 122 with which said retainer 118 was engaged. The inserter 102 may also be manipulated (e.g., twisted, pivoted, hinged, etc.) so as to release the staple 120 from the volume 116 (and the opposing end of the bridge 122 from the second retainer 118), thus permitting the staple 120 to return to the first position (in which the legs 124 form an oblique angle relative to one another and the bridge 122 has a substantially curved geometry). In this second position, the staple 120 may apply a compressive force through at least one of the bridge 122 and the legs 124.

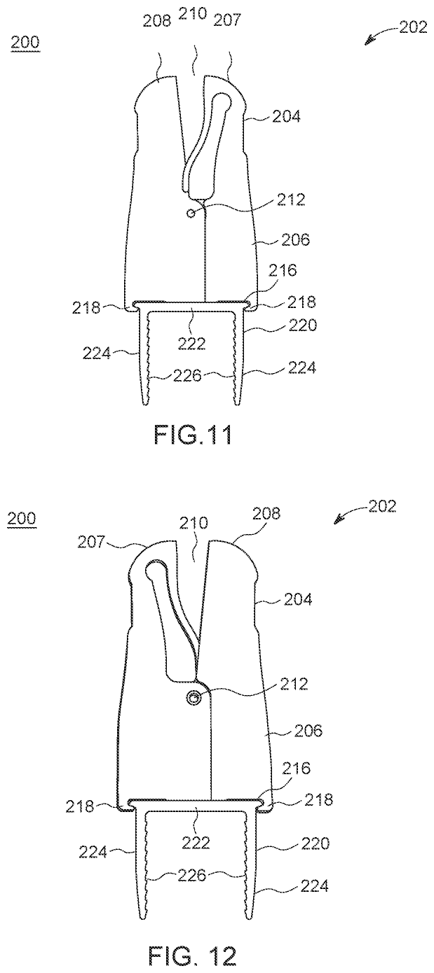

Referring now to FIGS. 11-12, an implant system 200 is shown, according to an exemplary embodiment. The implant system 200 may be variously sized, for example with one or more components configured in various sizes and the complementary component configured in a complementary size (e.g., a staple implant in various height/width configurations and an insertion instrument configured to releasably couple with said staple implant). Further, the implant system 200 may include cross-compatibly with one or more other systems shown and described herein, for example the system 100 and components thereof.

The implant system 200 is shown to include an insertion instrument 202 (referred to hereinafter as “inserter 202”), which is releasably couplable with an implant 220 (e.g., a staple) (referred to hereinafter as “staple 220”). In some aspects, the staple 202 may be the same as or similar to the staple 102. The inserter 202 and the staple 220 may be provided to a physician or other healthcare provider/facility in a coupled configuration (e.g., as shown in FIGS. 11-12), or may be provided in a decoupled configuration. If provided in a decoupled configuration, a physician or other healthcare provider may releasably couple (e.g., engage, etc.) the inserter 202 with the staple 220 prior to use or performing any procedure in which the implant system 200 and components thereof may be implemented. Further, the inserter 202 may be configured to releasably couple and decouple with multiple staples 202 in successive fashion, and as such may be configured to be a multi-use instrument. For example, a physician may releasably couple a first staple 220 with the inserter, decouple and implant said first staple 220, subsequently releasably couple a second staple 220 with the inserter 202, and ultimately decouple and implant said second staple 220.

The inserter 202 is shown to include an upper portion 204 configured substantially opposite a lower portion 206. As shown, the staple 220 is configured to releasably couple with a portion of the lower portion 206 of the staple 220, which is to say that the upper portion 204 is configured opposite the inserter 202 from the coupling site of the staple 220. In some aspects, one or both of the upper and lower portions 204, 206 of the inserter 202 may include one or more ergonomic features, for example geometric contours configured to facilitate gripping and manipulating/actuating the inserter 202 and components thereof. As shown, the upper and lower portions 204, 206 are integral with one another. The inserter 202 is further shown to include a first portion 207 and a second portion 208, with the first and second portions 207, 208 pivotable coupled to one another as a pivot point 212, as shown in FIGS. 11-12. The first and second portions 207, 208 are separated from one another (in the upper portion 204 of the inserter 202) by a volume 210 positioned therebetween and configured to be occupied by at least a portion of the first and/or second portions 207, 208 when manipulated relative to one another. In some aspects, the first portion 207 may be configured as an actuator (similar to the actuator 108) that can be manipulated to facilitate coupling/decoupling with the staple 220. In some aspects, a force may be required to manipulate the first portion 207 through a range of motion.

The lower portion 206 is further shown to include a volume 216, which is defined in an upward direction by the lower portion 206 (and bottom portions of both the first and second portions 207, 208) and in lateral directions by a pair of retainers 218. Each of the retainers 218 are shown to extend downward from a bottom portion of the lower portion 206, and are further shown to include substantially curved or “hooked” geometries with said curvature in the direction of a midline of the inserter 202 (e.g., toward the volume 216). The volume 216 as shown is not bound on a bottom side (e.g., opposite the portion defined by the bottom portion of the lower portion 206 when the inserter 202 is in an uncoupled state relative to the staple 220. As shown, the volume 216 is shown to have an elongated geometry (as defined on three sides), with a width dimension greater than a height dimension. As shown, said coupling includes positioning the bridge 222 of the staple 220 (in the aforementioned substantially straight geometry) at least partially within the volume 216 of the inserter 202.

The staple 220 is shown to include a pair of legs 224 extending from the bridge 222 at an angle substantially perpendicular to a bottom surface of the bridge 222. In the first aforementioned position, the legs 224 may be positioned at a substantially oblique angle relative to one another. However, in the second position (e.g., the position the staple 220 is retained in when coupled with the inserter 202), the legs 224 may be substantially parallel to one another. The legs 224 are further shown to include a texture 226 disposed on an inner surface thereof. As shown in FIGS. 11-12, the texture 226 includes a serration or toothed configuration, although in some aspects the texture 226 may also include alternate geometries and/or configurations.

The legs 224 extend downward from the bottom surface of the bridge 222 at points adjacent to the terminal ends of the bridge 222. However, each end of the bridge 222 is configured to extend laterally past the point from which the legs 224 extend. The aforementioned end portions of the bridge 222 (e.g., overhangs, shoulders, etc.) are configured to interface with the retainers 218 of the inserter 202 when in the coupled position. In coupling the staple 220 with the inserter 202, the staple may be manipulated to the second position (substantially straight/linear bridge), with the bridge 222 portion then inserted into the volume 216 such that the end portions of the bridge 222 abut the retainers 218 such that the retainers 218 exert a force on the end portions of the bridge 222 so as to retain the bridge 222 in the substantially straight/linear position.

Upon insertion, the first portion 207 of the inserter 202 may be manipulated such that at least a portion of the first portion 207 occupies at least a portion of the volume 210 and, accordingly, retainer 218 being integral with the first portion 207 disengages from the end portion of the bridge 222 with which said retainer 218 was engaged. The inserter 202 may also be manipulated (e.g., twisted, pivoted, hinged, etc.) so as to release the staple 220 from the volume 216 (and the opposing end of the bridge 222 from the second retainer 218), thus permitting the staple 220 to return to the first position (in which the legs 224 form an oblique angle relative to one another and the bridge 222 has a substantially curved geometry). In this second position, the staple 220 may apply a compressive force through at least one of the bridge 222 and the legs 224.

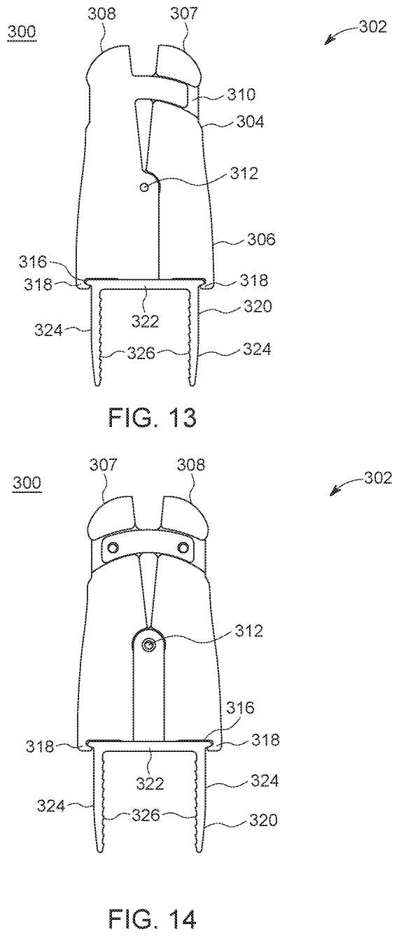

Referring now to FIGS. 13-14, an implant system 300 is shown, according to an exemplary embodiment. The implant system 300 may be variously sized, for example with one or more components configured in various sizes and the complementary component configured in a complementary size (e.g., a staple implant in various height/width configurations and an insertion instrument configured to releasably couple with said staple implant). Further, the system 300 may include cross-compatibly with one or more other systems shown and described herein, for example the systems 100, 200 and components thereof.

The system 300 is shown to include an insertion instrument 302 (referred to hereinafter as “inserter 302”), which is releasably couplable with an implant 320 (e.g., a staple) (referred to hereinafter as “staple 320”). In some aspects, the staple 302 may be the same as or similar to the staples 102, 202. The inserter 302 and the staple 320 may be provided to a physician or other healthcare provider/facility in a coupled configuration (e.g., as shown in FIGS. 13-14), or may be provided in a decoupled configuration. If provided in a decoupled configuration, a physician or other healthcare provider may releasably couple (e.g., engage, etc.) the inserter 302 with the staple 320 prior to use or performing any procedure in which the system 300 and components thereof may be implemented. Further, the inserter 302 may be configured to releasably couple and decouple with multiple staples 302 in successive fashion, and as such may be configured to be a multi-use instrument. For example, a physician may releasably couple a first staple 320 with the inserter, decouple and implant said first staple 320, subsequently releasably couple a second staple 320 with the inserter 302, and ultimately decouple and implant said second staple 320.

The inserter 302 is shown to include an upper portion 304 configured substantially opposite a lower portion 306. As shown, the staple 320 is configured to releasably couple with a portion of the lower portion 306 of the staple 320, which is to say that the upper portion 304 is configured opposite the inserter 302 from the coupling site of the staple 320. In some aspects, one or both of the upper and lower portions 304, 306 of the inserter 302 may include one or more ergonomic features, for example geometric contours configured to facilitate gripping and manipulating/actuating the inserter 302 and components thereof. As shown, the upper and lower portions 304, 306 are integral with one another. The inserter 302 is further shown to include a first portion 307 and a second portion 308, with the first and second portions 307, 308 pivotable coupled to one another as a pivot point 312, as shown in FIGS. 13-14. The first and second portions 307, 308 are separated from one another (in the upper portion 304 of the inserter 302) by a volume 310 positioned therebetween and configured to be occupied by at least a portion of the first and/or second portions 307, 308 when manipulated relative to one another. In some aspects, the first portion 307 may be configured as an actuator (similar to the actuator 108) that can be manipulated to facilitate coupling/decoupling with the staple 320. In some aspects, a force may be required to manipulate the first portion 307 through a range of motion.

The lower portion 306 is further shown to include a volume 316, which is defined in an upward direction by the lower portion 306 (and bottom portions of both the first and second portions 307, 308) and in lateral directions by a pair of retainers 318. Each of the retainers 318 are shown to extend downward from a bottom portion of the lower portion 306, and are further shown to include substantially curved or “hooked” geometries with said curvature in the direction of a midline of the inserter 302 (e.g., toward the volume 316). The volume 316 as shown is not bound on a bottom side (e.g., opposite the portion defined by the bottom portion of the lower portion 306 when the inserter 302 is in an uncoupled state relative to the staple 320. As shown, the volume 316 is shown to have an elongated geometry (as defined on three sides), with a width dimension greater than a height dimension. As shown, said coupling includes positioning the bridge 322 of the staple 320 (in the aforementioned substantially straight geometry) at least partially within the volume 316 of the inserter 302.

The staple 320 is shown to include a pair of legs 324 extending from the bridge 322 at an angle substantially perpendicular to a bottom surface of the bridge 322. In the first aforementioned position, the legs 324 may be positioned at a substantially oblique angle relative to one another. However, in the second position (e.g., the position the staple 320 is retained in when coupled with the inserter 302), the legs 324 may be substantially parallel to one another. The legs 324 are further shown to include a texture 326 disposed on an inner surface thereof. As shown in FIGS. 13-14, the texture 326 includes a serration or toothed configuration, although in some aspects the texture 326 may also include alternate geometries or configurations.

The legs 324 extend downward from the bottom surface of the bridge 322 at points adjacent to the terminal ends of the bridge 322. However, each end of the bridge 322 is configured to extend laterally past the point from which the legs 324 extend. The aforementioned end portions of the bridge 322 (e.g., overhangs, shoulders, etc.) are configured to interface with the retainers 318 of the inserter 302 when in the coupled position. In coupling the staple 320 with the inserter 302, the staple may be manipulated to the second position (substantially straight/linear bridge), with the bridge 322 portion then inserted into the volume 316 such that the end portions of the bridge 322 abut the retainers 318 such that the retainers 318 exert a force on the end portions of the bridge 322 so as to retain the bridge 322 in the substantially straight/linear position.

Upon insertion, the first and/or second portions 307, 308 of the inserter 302 may be manipulated such that at least a portion of the first and/or second portions 307, 308 occupies at least a portion of the volume 310 and, accordingly, retainer 318 integral with the first portion 307 disengages from the end portion of the bridge 322 with which said retainer 318 was engaged. The inserter 302 may also be manipulated (e.g., twisted, pivoted, hinged, etc.) so as to release the staple 320 from the volume 316 (and the opposing end of the bridge 322 from the second retainer 318), thus permitting the staple 320 to return to the first position (in which the legs 324 form an oblique angle relative to one another and the bridge 322 has a substantially curved geometry). In this second position, the staple 320 may apply a compressive force through at least one of the bridge 322 and the legs 324.

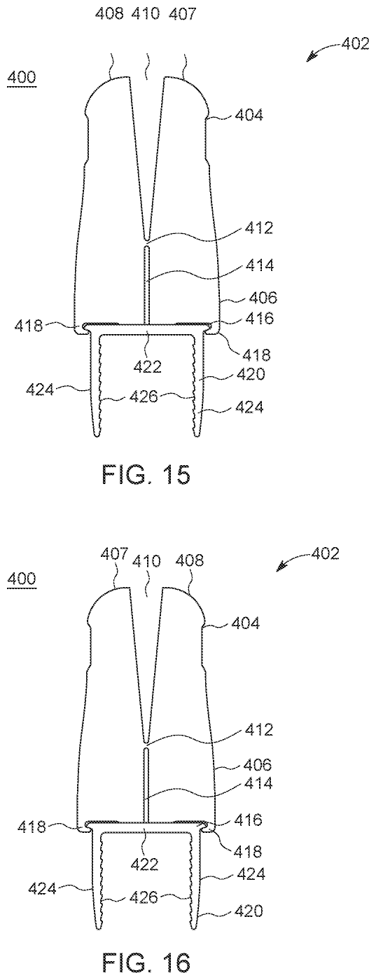

Referring now to FIGS. 15-16, an implant system 400 is shown, according to an exemplary embodiment. The implant system 400 may be variously sized, for example with one or more components configured in various sizes and the complementary component configured in a complementary size (e.g., a staple implant in various height/width configurations and an insertion instrument configured to releasably couple with said staple implant). Further, the system 400 may include cross-compatibly with one or more other systems shown and described herein, for example the systems 100, 200 and components thereof.

The system 400 is shown to include an insertion instrument 402 (referred to hereinafter as “inserter 402”), which is releasably couplable with an implant 420 (e.g., a staple) (referred to hereinafter as “staple 420”). In some aspects, the staple 402 may be the same as or similar to the staples 102, 202. The inserter 402 and the staple 420 may be provided to a physician or other healthcare provider/facility in a coupled configuration (e.g., as shown in FIGS. 15-16), or may be provided in a decoupled configuration. If provided in a decoupled configuration, a physician or other healthcare provider may releasably couple (e.g., engage, etc.) the inserter 402 with the staple 420 prior to use or performing any procedure in which the system 400 and components thereof may be implemented. Further, the inserter 402 may be configured to releasably couple and decouple with multiple staples 402 in successive fashion, and as such may be configured to be a multi-use instrument. For example, a physician may releasably couple a first staple 420 with the inserter, decouple and implant said first staple 420, subsequently releasably couple a second staple 420 with the inserter 402, and ultimately decouple and implant said second staple 420.

The inserter 402 is shown to include an upper portion 404 configured substantially opposite a lower portion 406. As shown, the staple 420 is configured to releasably couple with a portion of the lower portion 406 of the staple 420, which is to say that the upper portion 404 is configured opposite the inserter 402 from the coupling site of the staple 420. In some aspects, one or both of the upper and lower portions 404, 406 of the inserter 402 may include one or more ergonomic features, for example geometric contours configured to facilitate gripping and manipulating/actuating the inserter 402 and components thereof. As shown, the upper and lower portions 404, 406 are integral with one another. The inserter 402 is further shown to include a first portion 407 and a second portion 408, with the first and second portions 307, 408 pivotable coupled to one another as a pivot point 412, as shown in FIGS. 15-16. The first and second portions 407, 408 are separated from one another (in the upper portion 404 of the inserter 402) by a volume 410 positioned therebetween and configured to be occupied by at least a portion of the first and/or second portions 307, 408 when manipulated relative to one another. In some aspects, the first portion 407 may be configured as an actuator (similar to the actuator 108) that can be manipulated to facilitate coupling/decoupling with the staple 420. In some aspects, a force may be required to manipulate the first portion 407 through a range of motion.

The lower portion 406 is further shown to include a volume 416, which is defined in an upward direction by the lower portion 406 (and bottom portions of both the first and second portions 407, 408) and in lateral directions by a pair of retainers 418. Each of the retainers 418 are shown to extend downward from a bottom portion of the lower portion 406, and are further shown to include substantially curved or “hooked” geometries with said curvature in the direction of a midline of the inserter 402 (e.g., toward the volume 416). The volume 416 as shown is not bound on a bottom side (e.g., opposite the portion defined by the bottom portion of the lower portion 406 when the inserter 402 is in an uncoupled state relative to the staple 420. As shown, the volume 416 is shown to have an elongated geometry (as defined on three sides), with a width dimension greater than a height dimension. As shown, said coupling includes positioning the bridge 422 of the staple 420 (in the aforementioned substantially straight geometry) at least partially within the volume 416 of the inserter 402.

The staple 420 is shown to include a pair of legs 424 extending from the bridge 422 at an angle substantially perpendicular to a bottom surface of the bridge 422. In the first aforementioned position, the legs 424 may be positioned at a substantially oblique angle relative to one another. However, in the second position (e.g., the position the staple 420 is retained in when coupled with the inserter 402), the legs 424 may be substantially parallel to one another. The legs 424 are further shown to include a texture 426 disposed on an inner surface thereof. As shown in FIGS. 15-16, the texture 426 includes a serration or toothed configuration, although in some aspects the texture 426 may also include alternate geometries and configurations.

The legs 424 extend downward from the bottom surface of the bridge 422 at points adjacent to the terminal ends of the bridge 422. However, each end of the bridge 422 is configured to extend laterally past the point from which the legs 424 extend. The aforementioned end portions of the bridge 422 (e.g., overhangs, shoulders, etc.) are configured to interface with the retainers 418 of the inserter 402 when in the coupled position. In coupling the staple 420 with the inserter 402, the staple may be manipulated to the second position (substantially straight/linear bridge), with the bridge 422 portion then inserted into the volume 416 such that the end portions of the bridge 422 abut the retainers 418 such that the retainers 418 exert a force on the end portions of the bridge 422 so as to retain the bridge 422 in the substantially straight/linear position.

Upon insertion, the first and/or second portions 407, 408 of the inserter 402 may be manipulated such that at least a portion of the first and/or second portions 407, 408 occupies at least a portion of the volume 410 and, accordingly, retainer 418 integral with the first portion 407 disengages from the end portion of the bridge 422 with which said retainer 418 was engaged. The inserter 402 may also be manipulated (e.g., twisted, pivoted, hinged, etc.) so as to release the staple 420 from the volume 416 (and the opposing end of the bridge 422 from the second retainer 418), thus permitting the staple 420 to return to the first position (in which the legs 424 form an oblique angle relative to one another and the bridge 422 has a substantially curved geometry). In this second position, the staple 420 may apply a compressive force through at least one of the bridge 422 and the legs 424.

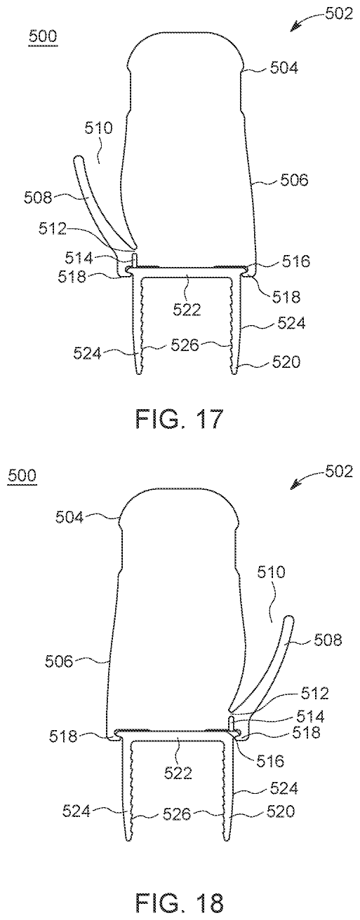

Referring now to FIGS. 17-18, an implant system 500 is shown, according to an exemplary embodiment. The implant system 500 may be variously sized, for example with one or more components configured in various sizes and the complementary component configured in a complementary size (e.g., a staple implant in various height/width configurations and an insertion instrument configured to releasably couple with said staple implant).

The system 500 is shown to include an insertion instrument 502 (referred to hereinafter as “inserter 502”), which is releasably couplable with an implant 520 (e.g., a staple) (referred to hereinafter as “staple 520”). The inserter 502 and the staple 520 may be provided to a physician or other healthcare provider/facility in a coupled configuration (e.g., as shown in FIGS. 17-18), or may be provided in a decoupled configuration. If provided in a decoupled configuration, a physician or other healthcare provider may releasably couple (e.g., engage, etc.) the inserter 502 with the staple 520 prior to use or performing any procedure in which the system 500 and components thereof may be implemented. Further, the inserter 502 may be configured to releasably couple and decouple with multiple staples 502 in successive fashion, and as such may be configured to be a multi-use instrument. For example, a physician may releasably couple a first staple 520 with the inserter, decouple and implant said first staple 520, subsequently releasably couple a second staple 520 with the inserter 502, and ultimately decouple and implant said second staple 520. Both the inserter 502 and staple 520 may be similar to and/or include cross-compatibility with systems and components shown and described previously herein.

The inserter 502 is shown to include an upper portion 504 configured substantially opposite a lower portion 506. As shown, the staple 520 is configured to releasably couple with a portion of the lower portion 506 of the staple 520, which is to say that the upper portion 504 is configured opposite the inserter 502 from the coupling site of the staple 520. In some aspects, one or both of the upper and lower portions 504, 506 of the inserter 502 may include one or more ergonomic features, for example geometric contours configured to facilitate gripping and manipulating/actuating the inserter 502 and components thereof. As shown, the upper and lower portions 504, 506 are integral with one another. However, in some aspects, the upper and lower portions 504, 506 may be releasably, hingedly, or otherwise coupled with one another.

The lower portion 506 is shown to include an actuator 508 extending laterally from a side of the lower portion 506. As shown, the actuator 508 is integral with the lower portion 506, but is movable relative to the lower portion 506. The actuator 508 includes an elongated geometry extending from the lower portion 506 and defining a volume (e.g., a space, opening, etc.) 510 positioned between at least a portion of the actuator 508 and the lower portion 506. The actuator 508 is manipulatable through a range of motion that includes at least the position shown in FIGS. 17-18 (an unactuated position), and a position in which at least a portion of the actuator 508 occupies at least a portion of the volume 510 (an actuated position). The actuator 508 may be formed from a resilient material and/or be structured such that a force must be applied to manipulate the actuator 508 from the unactuated position to the actuated position. Further, a force may be required to retain the actuator 508 in the actuated position, where absence of such a force may result in the actuator 508 returning to the unactuated position.

The actuator 508 is configured to be manipulatable about a pivot point 512 (referred to hereinafter as “point 512”) (or, in some aspects, a hinge point), where said point 512 is positioned at or adjacent to the point on the lower portion 506 as which the actuator 508 extends laterally. In some aspects, the point 512 may be configured on a portion of the lower portion that has a lesser width (e.g., lateral dimension) than the actuator 508. Further, the point 512 is positioned substantially below the volume 510 and above a volume 514, where the volume 514 is configured to receive at least a portion of the actuator 508 as said actuator 508 moves through the aforementioned range of motion.

The lower portion 506 is further shown to include a volume 516, which is defined in an upward direction by the lower portion 506 and in lateral directions by a pair of retainers 518. Each of the retainers 518 are shown to extend downward from a bottom portion of the lower portion 506, and are further shown to include substantially curved or “hooked” geometries with said curvature in the direction of a midline of the inserter 502 (e.g., toward the volume 516). The volume 516 as shown is not bound on a bottom side (e.g., opposite the portion defined by the bottom portion of the lower portion 506 when the inserter 502 is in an uncoupled state relative to the staple 520. As shown, the volume is shown to have an elongated geometry (as defined on three sides), with a width dimension greater than a height dimension.

As shown in FIGS. 17-18, the volume 516 is shown to receive a bridge 522 of the staple 520 when the staple 520 is in a coupled configuration with the inserter 502. In some aspects, the staple 502 may be manipulated from a first position (e.g., with the bridge 522 in a substantially curved geometry) to a second position (e.g., with the bridge 522 in a substantially straight geometry) prior to coupling with the inserter 502. In some aspects, a force may be required to manipulate the staple 502 and the bridge 522 thereof from the first position to the second position. As shown, said coupling includes positioning the bridge 522 of the staple 520 (in the aforementioned substantially straight geometry) at least partially within the volume 516 of the inserter 502.

The staple 520 is shown to include a pair of legs 524 extending from the bridge 522 at an angle substantially perpendicular to a bottom surface of the bridge 522. In the first aforementioned position, the legs 524 may be positioned at a substantially oblique angle relative to one another. However, in the second position (e.g., the position the staple 520 is retained in when coupled with the inserter 502), the legs 524 may be substantially parallel to one another. The legs 524 are further shown to include a texture 526 disposed on an inner surface thereof. As shown in FIGS. 17-18, the texture 526 includes a serration or toothed configuration, although in some aspects the texture 526 may also include alternate geometries and configurations.

The legs 524 extend downward from the bottom surface of the bridge 522 at points adjacent to the terminal ends of the bridge 522. However, each end of the bridge 522 is configured to extend laterally past the point from which the legs 524 extend. The aforementioned end portions of the bridge 522 (e.g., overhangs, shoulders, etc.) are configured to interface with the retainers 518 of the inserter 502 when in the coupled position shown in FIGS. 17-18. In coupling the staple 520 with the inserter 502, the staple may be manipulated to the second position (substantially straight/linear bridge), with the bridge 522 portion then inserted into the volume 516 such that the end portions of the bridge 522 abut the retainers 518 such that the retainers 518 exert a force on the end portions of the bridge 522 so as to retain the bridge 522 in the substantially straight/linear position.

Upon insertion, the actuator 508 of the inserter 502 may be manipulated such that at least a portion of the actuator 508 occupies at least a portion of the volume 510 and, accordingly, retainer 518 integral with the actuator 508 disengages from the end portion of the bridge 522 with which said retainer 518 was engaged. The inserter 502 may also be manipulated (e.g., twisted, pivoted, hinged, etc.) so as to release the staple 520 from the volume 516 (and the opposing end of the bridge 522 from the second retainer 518), thus permitting the staple 520 to return to the first position (in which the legs 524 form an oblique angle relative to one another and the bridge 522 has a substantially curved geometry). In this second position, the staple 520 may apply a compressive force through at least one of the bridge 522 and the legs 524.

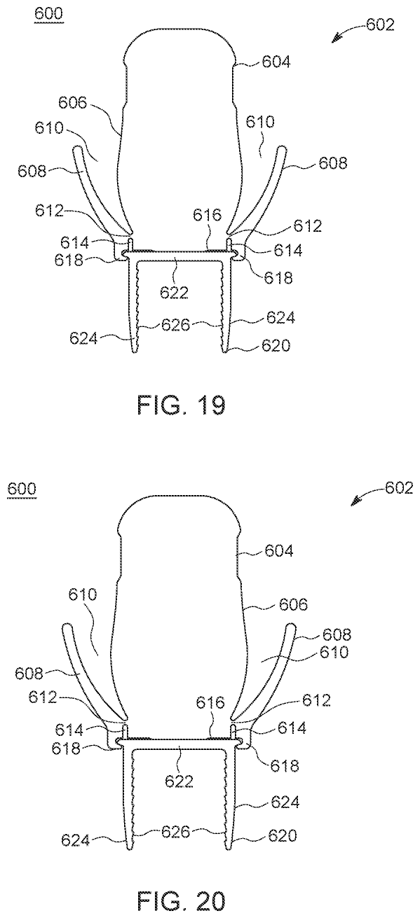

Referring now to FIGS. 19-20, an implant system 600 is shown, according to an exemplary embodiment. The implant system 600 may be variously sized, for example with one or more components configured in various sizes and the complementary component configured in a complementary size (e.g., a staple implant in various height/width configurations and an insertion instrument configured to releasably couple with said staple implant).

The system 600 is shown to include an insertion instrument 602 (referred to hereinafter as “inserter 602”), which is releasably couplable with an implant 620 (e.g., a staple) (referred to hereinafter as “staple 620”). The inserter 602 and the staple 620 may be provided to a physician or other healthcare provider/facility in a coupled configuration (e.g., as shown in FIGS. 19-20), or may be provided in a decoupled configuration. If provided in a decoupled configuration, a physician or other healthcare provider may releasably couple (e.g., engage, etc.) the inserter 602 with the staple 620 prior to use or performing any procedure in which the system 600 and components thereof may be implemented. Further, the inserter 602 may be configured to releasably couple and decouple with multiple staples 602 in successive fashion, and as such may be configured to be a multi-use instrument. For example, a physician may releasably couple a first staple 620 with the inserter, decouple and implant said first staple 620, subsequently releasably couple a second staple 620 with the inserter 602, and ultimately decouple and implant said second staple 620. Both the inserter 602 and staple 620 may be similar to and/or include cross-compatibility with systems and components shown and described previously herein.

The inserter 602 is shown to include an upper portion 604 configured substantially opposite a lower portion 606. As shown, the staple 620 is configured to releasably couple with a portion of the lower portion 606 of the staple 620, which is to say that the upper portion 604 is configured opposite the inserter 602 from the coupling site of the staple 620. In some aspects, one or both of the upper and lower portions 604, 606 of the inserter 602 may include one or more ergonomic features, for example geometric contours configured to facilitate gripping and manipulating/actuating the inserter 602 and components thereof. As shown, the upper and lower portions 604, 606 are integral with one another. However, in some aspects, the upper and lower portions 604, 606 may be releasably, hingedly, or otherwise coupled with one another.

The lower portion 606 is shown to include a pair of actuators 608 extending laterally from opposite sides of the lower portion 606. As shown, the actuators 608 are integral with the lower portion 606, but movable relative to the lower portion 606. The actuators 608 include an elongated geometry extending from the lower portion 606 and defining a pair of volumes (e.g., a space, opening, etc.) 610 positioned between at least a portion of the actuators 608 and the lower portion 606. The actuators 608 are manipulatable through a range of motion that includes at least the position shown in FIGS. 19-20 (an unactuated position), and a position in which at least a portion of the actuators 608 occupy at least a portion of the volumes 610 (an actuated position). The actuators 608 may be formed from a resilient material and/or be structured such that a force must be applied to manipulate the actuators 608 from the unactuated position to the actuated position. Further, a force may be required to retain the actuators 608 in the actuated position, where absence of such a force may result in the actuators 608 returning to the unactuated position.

The actuators 608 are configured to be manipulatable about a pair of pivot points 612 (referred to hereinafter as “points 612”) (or, in some aspects, a hinge point), where said points 612 are positioned at or adjacent to the point on the lower portion 606 as which the actuators 608 extend laterally. In some aspects, the points 612 may be configured on a portion of the lower portion that has a lesser width (e.g., lateral dimension) than the actuators 608. Further, the points 612 are positioned substantially below the volumes 610 and above a pair of volumes 614, where the volumes 614 are configured to receive at least a portion of the actuators 608 as said actuators 608 move through the aforementioned range of motion.

The lower portion 606 is further shown to include a volume 616, which is defined in an upward direction by the lower portion 606 and in lateral directions by a pair of retainers 618. Each of the retainers 618 are shown to extend downward from a bottom portion of the lower portion 606, and are further shown to include substantially curved or “hooked” geometries with said curvature in the direction of a midline of the inserter 602 (e.g., toward the volume 616). The volume 616 as shown is not bound on a bottom side (e.g., opposite the portion defined by the bottom portion of the lower portion 606 when the inserter 602 is in an uncoupled state relative to the staple 620. As shown, the volume is shown to have an elongated geometry (as defined on three sides), with a width dimension greater than a height dimension.

As shown in FIGS. 19-20, the volume 616 is shown to receive a bridge 622 of the staple 620 when the staple 620 is in a coupled configuration with the inserter 602. In some aspects, the staple 602 may be manipulated from a first position (e.g., with the bridge 622 in a substantially curved geometry) to a second position (e.g., with the bridge 622 in a substantially straight geometry) prior to coupling with the inserter 602. In some aspects, a force may be required to manipulate the staple 602 and the bridge 622 thereof from the first position to the second position. As shown, said coupling includes positioning the bridge 622 of the staple 620 (in the aforementioned substantially straight geometry) at least partially within the volume 616 of the inserter 602.

The staple 620 is shown to include a pair of legs 624 extending from the bridge 622 at an angle substantially perpendicular to a bottom surface of the bridge 622. In the first aforementioned position, the legs 624 may be positioned at a substantially oblique angle relative to one another. However, in the second position (e.g., the position the staple 620 is retained in when coupled with the inserter 602), the legs 624 may be substantially parallel to one another. The legs 624 are further shown to include a texture 626 disposed on an inner surface thereof. As shown in FIGS. 19-20, the texture 626 includes a serration or toothed configuration, although in some aspects the texture 626 may also include alternate geometries and configurations.

The legs 624 extend downward from the bottom surface of the bridge 622 at points adjacent to the terminal ends of the bridge 622. However, each end of the bridge 622 is configured to extend laterally past the point from which the legs 624 extend. The aforementioned end portions of the bridge 622 (e.g., overhangs, shoulders, etc.) are configured to interface with the retainers 618 of the inserter 602 when in the coupled position shown in FIGS. 19-20. In coupling the staple 620 with the inserter 602, the staple may be manipulated to the second position (substantially straight/linear bridge), with the bridge 622 portion then inserted into the volume 616 such that the end portions of the bridge 622 abut the retainers 618 such that the retainers 618 exert a force on the end portions of the bridge 622 so as to retain the bridge 622 in the substantially straight/linear position.

Upon insertion, one or both of the actuators 608 of the inserter 602 may be manipulated such that at least a portion of the actuator(s) 608 occupies at least a portion of the volume(s) 610 and, accordingly, retainer(s) 618 integral with the actuator(s) 608 disengage(s) from the end portion of the bridge 622 with which said retainer(s) 618 was engaged. The inserter 602 may also be manipulated (e.g., twisted, pivoted, hinged, etc.) so as to release the staple 620 from the volume 616 (and the opposing end of the bridge 622 from the second retainer 618), thus permitting the staple 620 to return to the first position (in which the legs 624 form an oblique angle relative to one another and the bridge 622 has a substantially curved geometry). In this second position, the staple 620 may apply a compressive force through at least one of the bridge 622 or the legs 624.

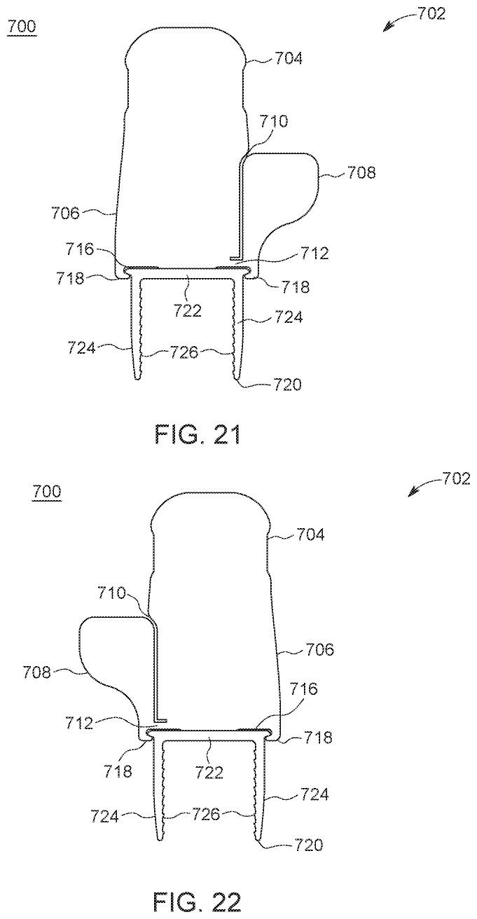

Referring now to FIGS. 21-22, an implant system 700 is shown, according to an exemplary embodiment. The implant system 700 may be variously sized, for example with one or more components configured in various sizes and the complementary component configured in a complementary size (e.g., a staple implant in various height/width configurations and an insertion instrument configured to releasably couple with said staple implant).

The system 700 is shown to include an insertion instrument 702 (referred to hereinafter as “inserter 702”), which is releasably couplable with an implant 720 (e.g., a staple) (referred to hereinafter as “staple 720”). The inserter 702 and the staple 720 may be provided to a physician or other healthcare provider/facility in a coupled configuration (e.g., as shown in FIGS. 21-22), or may be provided in a decoupled configuration. If provided in a decoupled configuration, a physician or other healthcare provider may releasably couple (e.g., engage, etc.) the inserter 702 with the staple 720 prior to use or performing any procedure in which the system 700 and components thereof may be implemented. Further, the inserter 702 may be configured to releasably couple and decouple with multiple staples 702 in successive fashion, and as such may be configured to be a multi-use instrument. For example, a physician may releasably couple a first staple 720 with the inserter, decouple and implant said first staple 720, subsequently releasably couple a second staple 720 with the inserter 702, and ultimately decouple and implant said second staple 720. Both the inserter 702 and staple 720 may be similar to and/or include cross-compatibility with systems and components shown and described previously herein.

The inserter 702 is shown to include an upper portion 704 configured substantially opposite a lower portion 706. As shown, the staple 720 is configured to releasably couple with a portion of the lower portion 706 of the staple 720, which is to say that the upper portion 704 is configured opposite the inserter 702 from the coupling site of the staple 720. In some aspects, one or both of the upper and lower portions 704, 706 of the inserter 702 may include one or more ergonomic features, for example geometric contours configured to facilitate gripping and manipulating/actuating the inserter 702 and components thereof. As shown, the upper and lower portions 704, 706 are integral with one another. However, in some aspects, the upper and lower portions 704, 706 may be releasably, hingedly, or otherwise coupled with one another.

The lower portion 706 is shown to include an actuator 708 extending laterally from a side of the lower portion 706. As shown, the actuator 708 is integral with the lower portion 706, but is movable relative to the lower portion 706. The actuator 708 includes a geometry increasing in width as the actuator 708 extends from the lower portion 706. As the actuator 708 extends from the lower portion 706, it defines a volume (e.g., a space, opening, etc.) 710 positioned between at least a portion of the actuator 708 and the lower portion 706. The actuator 708 is manipulatable through a range of motion that includes at least the position shown in FIGS. 21-22 (an unactuated position), and a position in which at least a portion of the actuator 708 occupies at least a portion of the volume 710 (an actuated position). The actuator 708 may be formed from a resilient material and/or be structured such that a force must be applied to manipulate the actuator 708 from the unactuated position to the actuated position. Further, a force may be required to retain the actuator 708 in the actuated position, where absence of such a force may result in the actuator 708 returning to the unactuated position.

The actuator 708 is configured to be manipulatable about a pivot point 712 (referred to hereinafter as “point 712”) (or, in some aspects, a hinge point), where said point 712 is positioned at or adjacent to the point on the lower portion 706 as which the actuator 708 extends laterally. In some aspects, the point 712 may be configured on a portion of the lower portion that has a lesser width (e.g., lateral dimension) than the actuator 708. Further, the point 712 is positioned substantially below the volume 710.

The lower portion 706 is further shown to include a volume 716, which is defined in an upward direction by the lower portion 706 and in lateral directions by a pair of retainers 718. Each of the retainers 718 are shown to extend downward from a bottom portion of the lower portion 706, and are further shown to include substantially curved or “hooked” geometries with said curvature in the direction of a midline of the inserter 702 (e.g., toward the volume 716). The volume 716 as shown is not bound on a bottom side (e.g., opposite the portion defined by the bottom portion of the lower portion 706 when the inserter 702 is in an uncoupled state relative to the staple 720. As shown, the volume is shown to have an elongated geometry (as defined on three sides), with a width dimension greater than a height dimension.

As shown in FIGS. 21-22, the volume 716 is shown to receive a bridge 722 of the staple 720 when the staple 720 is in a coupled configuration with the inserter 702. In some aspects, the staple 702 may be manipulated from a first position (e.g., with the bridge 722 in a substantially curved geometry) to a second position (e.g., with the bridge 722 in a substantially straight geometry) prior to coupling with the inserter 702. In some aspects, a force may be required to manipulate the staple 702 and the bridge 722 thereof from the first position to the second position. As shown, said coupling includes positioning the bridge 722 of the staple 720 (in the aforementioned substantially straight geometry) at least partially within the volume 716 of the inserter 702.

The staple 720 is shown to include a pair of legs 724 extending from the bridge 722 at an angle substantially perpendicular to a bottom surface of the bridge 722. In the first aforementioned position, the legs 724 may be positioned at a substantially oblique angle relative to one another. However, in the second position (e.g., the position the staple 720 is retained in when coupled with the inserter 702), the legs 724 may be substantially parallel to one another. The legs 724 are further shown to include a texture 726 disposed on an inner surface thereof. As shown in FIGS. 21-22, the texture 726 includes a serration or toothed configuration, although in some aspects the texture 726 may also include alternate geometries and configurations.

The legs 724 extend downward from the bottom surface of the bridge 722 at points adjacent to the terminal ends of the bridge 722. However, each end of the bridge 722 is configured to extend laterally past the point from which the legs 724 extend. The aforementioned end portions of the bridge 722 (e.g., overhangs, shoulders, etc.) are configured to interface with the retainers 718 of the inserter 702 when in the coupled position shown in FIGS. 21-22. In coupling the staple 720 with the inserter 702, the staple may be manipulated to the second position (substantially straight/linear bridge), with the bridge 722 portion then inserted into the volume 716 such that the end portions of the bridge 722 abut the retainers 718 such that the retainers 718 exert a force on the end portions of the bridge 722 so as to retain the bridge 722 in the substantially straight/linear position.

Upon insertion, the actuator 708 of the inserter 702 may be manipulated such that at least a portion of the actuator 708 occupies at least a portion of the volume 710 and, accordingly, retainer 718 integral with the actuator 708 disengages from the end portion of the bridge 722 with which said retainer 718 was engaged. The inserter 702 may also be manipulated (e.g., twisted, pivoted, hinged, etc.) so as to release the staple 720 from the volume 716 (and the opposing end of the bridge 722 from the second retainer 718), thus permitting the staple 720 to return to the first position (in which the legs 724 form an oblique angle relative to one another and the bridge 722 has a substantially curved geometry). In this second position, the staple 720 may apply a compressive force through at least one of the bridge 722 and the legs 724.

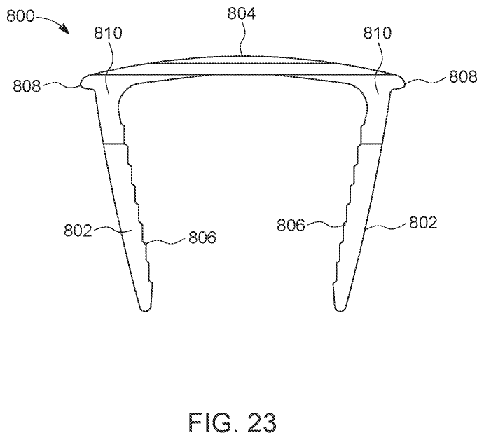

Referring now to FIGS. 23-30, an implant 800 (referred to hereinafter as “staple 800”) is shown, according to an exemplary embodiment. The staple 800 may be cross-compatible with one or more systems and/or components thereof shown and described previously herein. Further, the staple 800 may also be compatible with various other systems and components common to orthopedic procedures. In some aspects, the staple 800 may include features (including but not limited to those shown and described herein) which may facilitate said cross-compatibility via releasable coupling, engagement, actuation/manipulation, etc.

The staple 800 is shown to include a pair of legs 802 extending from a bridge 804, where said legs 802 form an oblique angle with one another when the staple is in the exemplary position of FIG. 23. The legs 802 are shown to be tapered along a length thereof, with the terminal end of the legs 802 having a lesser width/thickness than that of the portion adjacent the bridge 804. Each of the legs 802 are shown to extend from the bridge 804 from a point adjacent to the terminal ends of the bridge 804, thus defining a shoulder 808 at each end of the bridge 804 extending from the leg 802 to the terminal end of the bridge 804. The shoulders 808 may be configured to facilitate releasable coupling with an instrument, for example one or more of the inserters shown and described herein (i.e., 102, 202, 302, 402, 502 and 602). The legs 802 are shown to include a texture 806 disposed on at least an inner surface thereof, with said texture 806 shown in FIGS. 23-30 as a serrated, tooth-shaped geometry (although other textured geometries or configurations may be included in alternate embodiments).

The staple 800 is shown to include a transition 810 between the bridge 804 and each of the legs 802, where said transitions 810 are positioned between a midline of the staple 800 and the shoulders 808. The transitions 810 are shown to be radiused and, depending on the size of the staple 800, may vary in radius (where a greater radius would result in a lesser angle formed between the legs 802, and a lesser radius would result in a greater such angle).

The bridge 804 of the staple 800 is shown to include a top surface having a central portion 812 as well as lateral portions 814. The central portion 812 is positioned between the lateral portions 814, and extends along the entirety of the top surface of the bridge 804 from the shoulder 808 to the opposite shoulder 808. As shown, the central portion 812 is shown to have a substantially hourglass shape, with each of the lateral portions 814 disposed symmetrically on opposing sides of the central portion 812 and having substantially semi-circular shapes (as shown, less than half of a circle/elliptical shape).

The staple 800 may be configured to be manipulatable (e.g., moveable) between the first position of FIG. 23, and a second position in which the bridge 804 transitions from a substantially curved geometry to a substantially straight/linear geometry (and, in this position, the legs 802 no longer form an oblique angle with one another). The staple 800 may be retained in this second position by one or more inserter instruments including those shown and described herein previously. (i.e., 102, 202, 302, 402, 502 and 602). Further, the shoulders 808 may be configured to receive an applied force configured to bias the bridge 804 from the first position to the second position (and in some aspects, retain the bridge 804 in said second position).

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

What is claimed is:

1. An orthopedic implant system, comprising:
an insertion instrument comprising an actuator and a retention mechanism; and
an orthopedic implant configured to releasably couple with the retention mechanism of the insertion instrument and, upon manipulation of the actuator, decouple from the insertion instrument.

2. The implant system of claim 1, wherein the insertion instrument comprises:
an upper portion; and
a lower portion, wherein the upper and lower portions are integral with one another.

3. The implant system of claim 2, wherein the actuator is integral with the lower portion of the insertion instrument.

4. The implant system of claim 3, wherein the actuator extends laterally from the lower portion of the insertion instrument.

5. The implant system of claim 1, wherein the retention mechanism extends from the lower portion of the insertion instrument and comprises:
a pair of retainers.

6. The implant system of claim 5, wherein each retainer of the pair of retainers are positioned at an opposite side of the bottom portion of the insertion instrument.

7. The implant system of claim 6, wherein the insertion instrument comprises:
a volume defined vertically by the lower portion of the insertion instrument and laterally by each of the retainers.

8. The implant system of claim 7, wherein each of the retainers comprises:
a substantially curved geometry, wherein said curved geometry extends toward the volume.

9. The implant system of claim 8, wherein the volume is configured to receive and retain at least a portion of the implant therein.

10. The implant system of claim 9, wherein manipulation of the actuator is configured to manipulate at least one of the retainers so as to couple and decouple the implant from the insertion instrument.

11. The implant system of claim 1, wherein the implant comprises:
an orthopedic staple.

12. The implant system of claim 11, wherein the staple comprises:
a bridge portion comprising a pair of shoulders disposed at opposite ends of the bridge portion; and
a pair of legs extending from the bridge portion, with each leg positioned between one of the shoulders and a midline of the bridge portion.

13. A surgical instrument, comprising:
an upper portion;
a lower portion integral with an opposite the upper portion, the lower portion comprising:
an actuator extending laterally from the lower portion; and
a pair of retainers extending from a bottom portion of the lower portion toward the upper portion, wherein each retainer of the pair of retainers comprises a substantially curved geometry with said curvature pointing toward a midline of the instrument.

14. The instrument of claim 13, further comprising:
a first volume defined at least partially by a lateral surface of the instrument and a lateral surface of the actuator.

15. The instrument of claim 14, wherein the actuator is manipulatable such that at least a portion of the actuator occupies at least a portion of the first volume.

16. The instrument of claim 15, wherein the actuator is pivotable relative to the lower portion of the instrument.

17. The instrument of claim 16, wherein manipulation of the actuator alters the lateral dimension of a second volume defined laterally by the retainers.

18. An orthopedic staple implant, comprising:
a bridge;
a pair of legs extending from a bottom surface of the bridge, wherein each of the legs comprise a texture on an inner surface thereof and comprise a terminal end.

19. The implant of claim 18, wherein the implant is movable between a first position comprising the bridge in a substantially curved configuration, and a second position comprising the bridge in a substantially linear configuration.

20. The implant of claim 18, wherein each of the legs extend from the bottom surface of the bridge at a point between a midline of the bridge and a terminal end point of the bridge.

21. The implant of claim 20, wherein each of the legs comprise:
a tapered thickness, wherein said thickness is greater at a point adjacent the bridge than at a point adjacent the terminal end of the legs.
